(19) United States
(12) Patent Application Publication
(10) Pub. No.: US 2009/0017547 A1
(43) Pub. Date: Jan. 15, 2009

(54) SYSTEMS AND METHODS FOR DNA COMPUTING USING METHYLATION

(75) Inventors: Nevenka Dimitrova, Pelham Manor, NY (US); Susannah Gal, Binghamton, NY (US)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/279,874

(22) PCT Filed: Feb. 6, 2007
(86) PCT No.: PCT/IB07/50390

§ 371 Date: Aug. 19, 2008

Related U.S. Application Data
(60) Provisional application No. 60/776,758, filed on Feb. 24, 2006.
(60) Provisional application No. 60/805,778, filed on Jun. 26, 2006.

(51) Int. Cl. G01N 33/48; G06N 3/12

(52) U.S. Cl. 436/94; 706/13

(57) ABSTRACT

The present disclosure is directed to new methods and systems performing flexible and reversible modification of DNA in order to establish the logical value of true or false for a set of clauses. It combines both the biological meaning and experimental procedure with the logical implementation of the basic Boolean operators: OR, AND, and NOT. A feature of methylation logic is the use of the reversibility of DNA methylation of cytosine and adenine. Logic variables can be negated by reversing the DNA methylation status. Four implementation scenarios are described: three of them use methyl-sensitive restriction enzymes and the fourth uses methyl-binding proteins. Encoding can use either single or double stranded DNA. In addition, the disclosure allows for solving a multi-variable SAT problems by implementing a logic circuit.

### CROSS REFERENCE TO RELATED CASES

Applicants claim the benefit of Provisional Application Ser. No. 60/776,758, filed Feb. 24, 2006.

The present disclosure relates to systems and methods validating existing biomarkers and finding new biomarkers using DNA methylation. The disclosed systems and methods are useful in a variety of applications, including molecular diagnostics, DNA hypothesis generation, and in vitro experimentation.

DNA computing uses DNA and molecular biology, instead of the traditional silicon-based computer technologies to solve complex problems. A single gram of DNA with volume of 1 cm³ can hold as much information as a trillion compact discs, approximately 750 terabytes.

This field was initially developed by Leonard Adleman of the University of Southern California. In 1994, Adleman demonstrated a proof-of-concept use of DNA as a form of computation which was used to solve the seven-point Hamiltonian path problem. Since the initial Adleman experiments, advances have been made, and various Turing machines have been proven to be constructible. In 2004, Ehud Shapiro and researchers at the Weizmann Institute announced in the journal NATURE that they had constructed a DNA computer. The DNA computer was coupled with an input and output module and was capable of diagnosing cancerous activity within a cell, and then releasing an anti-cancer drug upon diagnosis. DNA computing is fundamentally similar to parallel computing, in that it takes advantage of the many different molecules of DNA to try many different possibilities at once.

DNA methylation refers to techniques that involve adding a methyl group, CH₃, to the fifth carbon on cytosine or to the sixth carbon of adenine. DNA methylation is a mechanism known both in animals and plants as an important means for gene expression regulation. In bacteria, it acts as a protection mechanism for protecting against attack by foreign DNA. As a biological process, DNA methylation is reversible. DNA methyltransferases catalyze the transfer of a methyl group from S-adenosyl-L-methionine to cytosine or adenine bases in DNA. DNA polymerases do not copy the methylated status during replication.

Certain assays known in the art are used as experimental tools for analysis in the field of developmental biology and cancer for DNA computation. These assays are primarily used for finding an epigenetic or methylation state of candidate genes and the involvement of the candidate gene(s) in certain biological process(es). These techniques can lead to identifying or verifying existing biomarkers or establishing new ones. Biomarkers play a significant role in distinguishing between cancerous and healthy cells via the DNA computing models. Currently, aqueous computing is limited in this field due to the inability to rewrite or overwrite on existing DNA. This limitation thwarts aggressive research, characterization of genetic sequences, highly complex problem solving, and diagnostic viability. Accordingly, once a piece of DNA has been processed, it is no longer reusable. Existing assay methods are irreversible, limiting their practical and informative value.

Molecular computing models have approached different NP-complete problems since Adleman's historical experiment in 1994. (See, e.g., Conrad, M., Information processing in molecular systems. Currents in Modern Biology, 1972. 5: p. 1-14, Livstone M S, v. N. D., Landweber L F., Molecular computing revisited: a Moore's Law? Trends Biotechnol., 2003. 21(3): p. 98-101, Head, T., One Mathematician's Tour from Biology into Computing and Back to Life. submitted, 2006). Lederman et al. developed an array of seven deoxyribozyme-based molecular logic gates that behave as a full adder in a single solution. (See, e.g., Lederman H, M. J., Stefanovic D, Stojanovic M N., Deoxyribozyme-Based Three-Input Logic Gates and Construction of a Molecular Full Adder. Biochemistry, 2006, Margolin A A, S. M., Boolean calculations made easy (for ribozymes). Nat Biotechnol., 2005). Liu et al. disclosed using a surface chemistry-based DNA computer to solve a four-variable four-clause 3-SAT problem. (See, e.g., Liu, Q., Wang, L., Frutos, A., Condon, A. E. and Smith, L. M., DNA computing on surfaces. Nature, 2000).

More recently, Su et al. have implemented a DNA computer capable of simulating Boolean logic circuits. (See, e.g., Su X, S. L., Demonstration of a universal surface DNA computer. Nucleic Acids Res., 2004). They constructed NOR and OR gates and combined them into a simple logic circuit. Head et al. proposed a novel way for recording information on DNA molecules while dissolved in water. (See, e.g., T. Head, X. C., M. J. Nichols, M. Yamamura, S. Gal, Aqueous solutions of algorithmic problems: emphasizing knights on a 3×3 in DNA Computing—7th International Workshop on DNA-Based Computers, 2002). The resulting solution containing molecules is considered to constitute a “fluid memory.” Head et al. also introduced schemes for reading information from these molecules.

Hatada et al. proposed a simple instance of the Satisfiability Problem of a set of Boolean Clauses (SAT problem). (Hatada I., F. M., Kimura M., Morita S., Yamada K., Yoshikawa T., Yamanaka S., Endo C., Sakurada A., Sato M., Kondo T., Horii A., Ushijima T., Sasaki H., Genome-wide profiling of promoter methylation in human, Oncogene, 2006). Given a set of Boolean clauses, the problem was to find truth values for which all of the clauses are satisfied (true). A procedure for solving this SAT problem illustrates the DNA computing method called the ‘Aqueous Algorithm’. (See, e.g., T. Head, X. C., M. Yamamura, S. Gal, Aqueous computing: a survey with an invitation to participate, J. Computer Science & Technology, 2002).

Benenson et al. implemented an automation in which computation is performed by a reversible software molecule with input molecule hybridization followed by an irreversible software-directed cleavage of the input molecule. (See, e.g., Benenson Y. A. R., Paz-Elizur T, Livneh Z, Shapiro E., DNA molecule provides a computing machine with both data and fuel Proc Natl Acad Sci U S A, 2003). Previously, Gal et al. took the approach of unidirectional methylation of specific restriction enzyme sites used to solve or model a specific SAT problem. (See, e.g., Gal S., H. T. Exploring Methylation as a Tool for DNA Computing in DNA11: Conference on DNA based computers. 2005).

Despite efforts to date, a need remains for systems and methods that effectively solve complex problems through reversible DNA computation. In addition, a need remains for systems/methods that are effective and reliable in achieving practical and viable diagnostic tools using methylation. These and other needs are satisfied by the systems and methods disclosed herein.

The present disclosure describes methods and systems for utilizing methylation logic for DNA computing. Methods and systems for verifying biomarkers using methylation logic are also disclosed. In an exemplary embodiment, the disclosed methods and systems involve generating a logic statement having assigned variables. The variables include a plurality of methylated variables and, for each such methylated variable, a negation thereof corresponding to an unmethylated variable. Once variables are assigned and a given series of logic clauses are chosen to determine a ‘True” value for the clause, the variables/logic clauses are correlated relative to a sample/mixture of interest.

Thus, according to exemplary embodiments of the present disclosure, a sample or mixture is obtained, identified and/or created. The mixture/sample is then methylated. Through one or more separation techniques, typically a series of separation techniques, the mixture/sample is separated through a series of assays. Through the noted separation technique(s), a final desired mixture/sample is obtained, which is then decoded using a decoding means. The decoded information is then generally read. In exemplary embodiments of the present disclosure, the reading can be used to verify an existing biomarker or to create a new one.

The variables associated with the logic statements of the disclosed systems and methods are typically a specified gene, but can also be a set of genes, a set of sites on a gene, and combinations thereof. The variables typically have at least one cytosine or adenine to accommodate/facilitate either a C-implementation methylation encoding or an A-implementation methylation encoding. Variable encoding can be accomplished according to the disclosed systems by single-strand or double-strand DNA.

A further aspect of the present disclosure relates to methods and systems for solving a set of genetic clauses, the methods/systems involving the assignment of variables such that each assigned variable corresponds to a methylated variable and a negation thereof—the negation corresponding to an unmethylated variable. The assignment of such variables allows for the solving for AND, OR, and NOT Boolean logic terms within a given logic clause, i.e., utilizing the methylated and unmethylated variables. The disclosed methods and systems generally include the steps of methylating a given mixture/sample containing components/constituents that correspond to the assigned variables, then separating the mixture/sample through one or more separation steps, e.g., a series of assays, to yield a desired mixture. According to exemplary embodiments of the present disclosure, the desired mixture satisfies the given logical clause. Accordingly, the assigned variables typically correspond to a specified gene, a set of genes, a set of sites on a gene, or the like. The variables typically have at least one cytosine to accommodate/facilitate C-implementation methylation encoding or at least one adenine to accommodate/facilitate A-implementation methylation encoding. Variable encoding can be accomplished by single-strand or double-strand DNA.

The disclosed methods and systems offer significant advantages for DNA computing. As such, the disclosed methods and systems have wide ranging applicability. Additional features, functions and benefits of the disclosed methods and systems will be apparent from the description which follows, particularly when read in conjunction with the appended figures.

To assist those of ordinary skill in the art in making and using the disclosed systems and methods, reference is made to the appended figure, wherein:

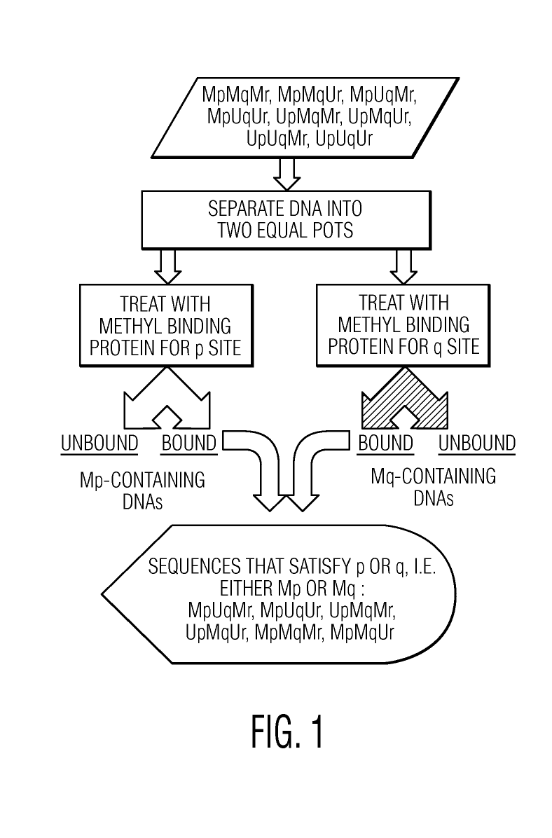

FIG. 1 is an illustration demonstrating the steps in computing p OR q.

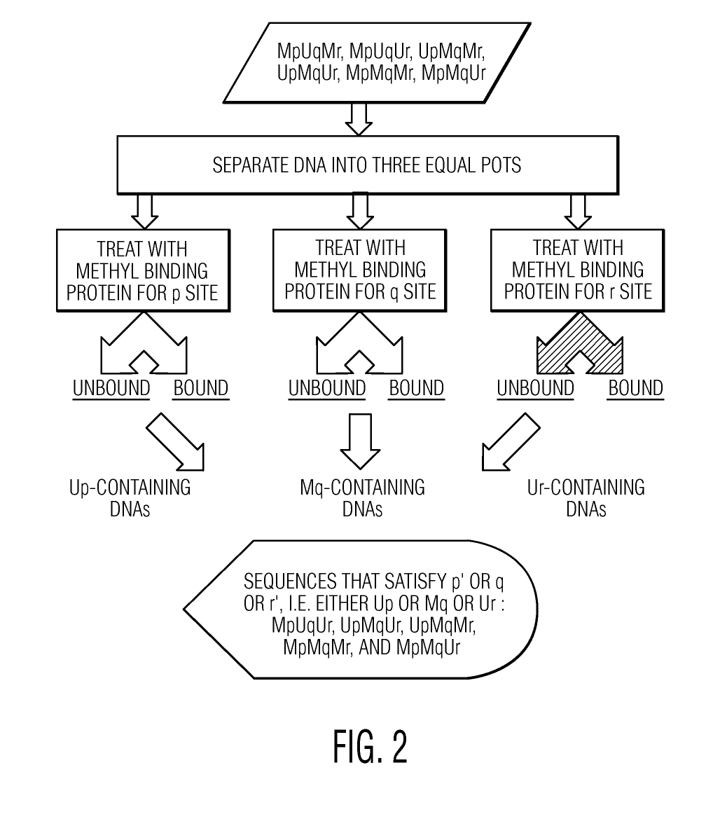

FIG. 2 is an illustration demonstrating the steps in computing p′ OR q OR r′.

The current disclosure describes a mathematical logic framework that advantageously allows for more complex operations on DNA in the process of making diagnostic decisions. As opposed to conventional DNA computing methods, the methods and systems of the present disclosure allow/facilitate writing and re-writing for DNA computations.

The disclosed methods and systems employ methylation logic, thereby utilizing the reversibility of DNA methylation of cytosine and/or adenine to support complex/advantageous computational techniques. The disclosed approach allows/facilitates reversible methylation of DNA sequences to change the truth value of encoded variables. The encoded variables include, but are not limited to, genes, sets of genes, and/or a section of a gene.

In a particular approach, methylation-sensitive restriction enzymes or methyl-binding proteins can be used to methylate cytosine. The DNA sequences encoding the “true” and “false” values of a particular logic variable do not have to be encoded with different sequences. Instead, the negation of a variable is encoded with the opposite state. For example, if a variable has a value of T and is encoded with an unmethylated sequence, then the negation of this variable is encoded with the same DNA sequence but methylated.

Two nucleotides exist that yield the methylation mark, namely, adenine and cytosine. Methylation logic implementations that are based on adenine and cytosine are called A-implementation and C-implementation, respectively. The examples set forth below provide exemplary implementations of a C-implementation to more clearly describe the present disclosure. However, as will be readily apparent to persons skilled in the art, the methods and systems of the present disclosure are not limited to C-implementation, but have wider applicability, e.g., to A-implementation.

The following describes a number of commercially available, exemplary biochemical tools suitable for applying a MethyLogic approach to computation, as disclosed herein. These tools are primarily used in conjunction with a computer/processor and the procedures described herein are performed in silica. Methylated DNA's of a specific sequence can be prepared simply by ordering oligonucleotides and requesting specific nucleotides as methyl-cytosine. An exemplary supplier is Integrated DNA Technologies, http://www.idtdna.com. There are several methylation transferases, methyl binding proteins and methyl-specific restriction enzymes known in the art that are capable of methylating a sequence, each of which may be used in connection with the disclosed methods/systems, either alone or in combination.

According to further exemplary embodiments of the present disclosure, a variety of enzymes exist that can methylate DNA at specific 4-6 base pair recognition sites. The human Dnmt1 enzyme methylates the cytosine in the C-G context, but only if one strand is already methylated (called hemi-methylated) to make it fully methylated on both strands. At present, more than thirteen (13) different DNA methylases are commercially available. Methylated DNA binding proteins can be used to physically separate methylated from unmethylated DNA. Alternative separation techniques may also be employed, either alone or in combination with the binding protein-based techniques. Several different binding proteins are known and suitable for use according to the present disclosure, including but not limited to: Kaiso, MBD1, MBD2, MBD3, MBD4, and MeCP. One or more of the foregoing binding proteins may be sequence specific, in which case utilization for such sequence(s) is generally effective.

It is also possible according to the present disclosure to carry out specific biochemical reactions to distinguish methylated from unmethylated DNA. For example, bisulfite treatment modifies unmethylated cytosines and converts them to uridine residues. Methylated cytosines are unmodified. Thus, a bisulfite treatment may be employed to create a single base mismatch between a uridine on one strand and a guanine on the other. A few specific endonucleases are available and known in the art that can cleave this structure specifically.

Alternatively, DNA sequencing, oligonucleotide hybridization or PCR can be used to distinguish different levels of methylation status of sequences. Recently, for example, a specific DNA endonuclease, McrBC, has been isolated that cuts hemi-methylated or methylated DNA. Thus, endonuclease compounds of the type characterized by McrBC may be employed to screen for methylated DNA sequences in human DNA. There are also sequence-specific DNA cleavage enzymes, restriction endonucleases, that can cleave depending on the methylation status of the DNA (for example, MspI and HpaII). When a pair of these enzymes is used with the same sequence, i.e., a first enzyme that can cut methylated DNA and the other that can not, comparison of the cleavage status in each reaction can be used according to the present disclosure to determine whether a specific DNA is methylated or not, even in a complicated mixture such as the human genome. These known tools are available for the analysis of logic algorithms using DNA methylation according to the methods and systems of the present disclosure.

In an exemplary embodiment of the present disclosure, Boolean logic using DNA methylation is advantageously employed for DNA computation. Since DNA methylation is a reversible process, it allows for an abstract framework. Indeed, a variety of physical implementations are available, thereby yielding in a plurality of potential implementation procedures that give substantial freedom in DNA selection. DNA methylation is important because the write-erase steps can be implemented as methylate-unmethylate in solution. Methylation logic that allows/facilitates the use of differently encoded strings is defined by the present disclosure. A general requirement is that encoded logical variables contain at least one cytosine for C-implementation or at least one adenine for A-implementation. According to the present disclosure, one of the DNA methylation states is taken as true while the other methylation state is taken as false. For example, methylation of cytosine may be taken as equivalent to “True” for a given variable.

The following definitions/description are provided in connection with the present disclosure.

Encoding: Logic variables can be encoded using single or double stranded DNA. In the C-implementation, the codes typically include CpG dinucleotide.

Write: Writing corresponds to applying DNA methylation either in vitro or in vivo. According to exemplary embodiments of the present disclosure, in vitro methylation corresponds to applying one of the methyl-transferase enzymes previously described. In vivo methylation may correspond to a maintenance methyltransferase DNMT1 which methylates C within a CpG dinucleotide only if one of the strands is already methylated and de novo methyltransferases DNMT3a and DNMT3b methylate all the CpG dinucleotides.

Erase: Erasing corresponds to any procedure previously described that removes DNA methylation mark in vitro or in vivo.

Destroy: Any procedure that involves destroying unmethylated or methylated DNA is encompassed within the term “destroy.” For example, destroying may involve applying one or more enzymes that digest specifically methylated or unmethylated DNA. Procedures such as PCR that lead to the loss of the methylation mark are a further example for purposes of the present disclosure.

Separate: A variety of techniques may be employed to separate methylated and unmethylated constituents according to the present disclosure. For example, methylated DNA binding proteins can be used to separate strands of DNA that have methylated nucleotides from those without any methyl groups attached.

Read: As used herein, the term “read” refers to a technique or system that may be used to generate a readout procedure or other indicia that can distinguish if a piece of DNA is fully, hemi, or partially methylated or completely unmethylated. Methylation-sensitive restriction enzymes, and PCR can be used for this purpose.

In any computation process, a duality may exist between encoding/reading procedure(s) vs. computation procedure(s). Typically, a computation procedure uses various physical and chemical processes, thereby generating results for the reading procedure to analyze and/or interpret. The present disclosure describes four (4) exemplary implementation scenarios; the first three exemplary scenarios can be implemented using methyl-sensitive restriction enzymes and the fourth implementation scenario uses methyl-binding proteins. Described is implementation of AND and OR logical operators. Implementation of NOT is by reversing the methylation status of the input sequence (variable). This could be done with the “write” and “erase” processes mentioned above.

### Implementation Case 1:

Encoding: Sequences are encoded with single-stranded DNA, the “logical operators” are evaluated after allowing sequences to hybridize;

Boolean terms

AND: both strands and all CpG sites should be methylated to have a truth value of “True” else the truth value is “False”;

OR: hemi-methylated or fully methylated DNA are treated as “True” whereas unmethylated DNA is treated as “False.”

Single-stranded can come from two different double stranded regions that have been melted and re-hybridized. For example, take paternal and maternal chromosomes then melt them and allow them to rehybridize which would form a hybrid chromosome with one strand from the paternal and one strand from the maternal chromosome.

### Implementation Case 2:

Encoding: Sequences are encoded as double-stranded DNA, the operation is the same for AND and OR, but the readout is interpreted/analyzed differently based on intended operator. New sequences are ligated from existing ones in order to make logical propositions ( or circuits):

Boolean terms

AND: requires the entire length of the sequence to be methylated to have “True”, else it is “False”. Of course, both strands have to continue to be methylated.

OR: requires any region to be methylated to have “True” else it is “False.”

### Implementation Case 3:

This third exemplary implementation involves a combination of the foregoing implementation cases 1 and 2, where single stranded DNA represents logical variables, and ligating double stranded DNA is used to implement complex logical expressions.

### Implementation Case 4:

Encoding: Logic variables are encoded as single or double stranded DNA. Using methyl binding proteins including methyl specific antibodies (or other separation technique), double-stranded DNA can be separated into a “bound” fraction (having methylated DNA) and an “unbound” fraction (having only unmethylated DNA). Thus, in exemplary techniques where methyl-binding proteins are employed for separation purposes, encoded sequences are allowed to hybridize and then methyl-binding proteins are used to fish out any DNA sequence that has methylation. Using PCR, it is possible to distinguish in a sensitive and sequence-specific manner whether sequences are in the bound or unbound fraction or both. With less complicated mixtures, it is possible to see the separation on a gel. If implementing logical variables that involve representations from the human genome is desired, then PCR may be advantageously used to see in which fraction a given sequence is present.

Boolean terms

AND: if the DNA sequences are both in the “bound” fraction, the truth value is “True”. Otherwise, it evaluates to “False”.

OR: if either DNA sequence is in the “bound” fraction, meaning that at least some of the DNA sequence is methylated, it is evaluated to “True”. If both DNA sequences are in the unbound fraction, meaning that the DNA sequence is unmethylated, it is evaluated to “False”.

The following describes how methylation logic using Single Stranded DNA (ssDNA) can be achieved according to exemplary embodiments of the present disclosure:

Logical Operator AND using ssDNA

Table 1 shows Boolean logic and methylation logic equivalent for the logical operator AND. The logical variables are encoded as single-stranded DNA converted to double-stranded DNA by hybridizing the strands. In the present disclosure, A and B are two single-stranded DNA hybridized together or are two different sites on double-stranded DNA. The truth value of the hybridized product is “True” if and only if the double-stranded DNA is methylated on both strands. Alternatively, the logical variables are encoded as two different sites on the now double-stranded DNA. If both sites are methylated, then the truth value is “True.” There are various implementation considerations to be made. In certain embodiments of the present disclosure, implementation of an AND term may require an experimental procedure to verify for full methylation. In exemplary embodiments of the present disclosure, applying HpaII digestion is completed to maintain intact only completely methylated DNA. This restriction enzyme is sensitive to methylation and thus can not cut methylated DNA. To distinguish hemi-methylated from methylated DNA, the bisulfite treatment may be applied first, followed by using enzymes that cut at a mismatch. A bisulfite treatment may be used to convert an unmethylated-C to a U, thus creating a mis-paired base with the G on the opposite strand. Those mis-paired bases can then be cut with the specific enzymes recognizing the mismatch. This protocol should yield only intact fully methylated DNA.

TABLE 1
Methylation logic table for AND operator.

| Boolean Logic | | | MethyLogic | | | |
|---|---|---|---|---|---|---|
| A | B | A AND B | A | B | A AND B | Realization |
| T | T | T | M | M | M | fully methylated |
| | | | | | | on both strands |
| | | | | | | or at both sites |
| T | ⊥ | ⊥ | M | U | U | unmethylated at |
| | | | | | | least on one |
| | | | | | | strand (hemi- |
| | | | | | | methylated DNA) |
| | | | | | | or site |
| ⊥ | T | ⊥ | U | M | U | unmethylated at |
| | | | | | | least on one |
| | | | | | | strand (hemi- |
| | | | | | | methylated DNA) |
| | | | | | | or site |
| ⊥ | ⊥ | ⊥ | U | U | U | fully unmethylated |
| | | | | | | on both strands or |
| | | | | | | at both sites |

Logical Operator OR using ssDNA

Table 2 shows the Boolean logic and methylation logic equivalent for the logical operator OR. The logical variables are encoded as single-stranded DNA, then converted to double-stranded DNA using hybridization. The truth value of the hybridized product is equal to “True” if the double stranded DNA is methylated on at least one strand. Alternatively, variables A and B can represent two different sites on a double-stranded DNA molecule. When either site is methylated, the resulting truth value is “True”.

TABLE 2
Methylation logic table for OR operator.

| Boolean Logic | | | MethyLogic | | | |
|---|---|---|---|---|---|---|
| A | B | A OR B | A | B | A OR B | Realization |
| T | T | T | M | M | M | fully methylated on both |
| | | | | | | strands or at both sites |
| T | ⊥ | T | M | U | M | unmethylated at least on |
| | | | | | | one strand (hemi-methylation) |
| | | | | | | or at one site |
| ⊥ | T | T | U | M | M | unmethylated at least on |
| | | | | | | one strand (hemi-methylation) |
| | | | | | | or at one site |
| ⊥ | ⊥ | ⊥ | U | U | U | fully unmethylated on both |
| | | | | | | strands or at both sites |

As in the case of AND, A and B are two single-stranded DNA hybridized together or are two different sites on double-stranded DNA. In certain embodiments of the present disclosure, implementation of the OR term may require an experimental procedure to verify whether a sequence is hemi- or fully methylated. MCrBC enzyme may be applied to cut all methylated or hemi-methylated sequences. This enzyme application keeps intact only the unmethylated sequences. Alternatively, methyl binding proteins or other separation technique can be used, as mentioned in implementation scenario 4, to fish out anything that has methylation. The unmethylated DNA would be in the unbound portion.

Logical Operator NOT using ssDNA

Table 3 shows the Boolean logic and methylation logic equivalent for the logical operator NOT. A logical variable is encoded as single stranded DNA. The truth value is reversed by using PCR if the sequence is methylated because, during PCR, the methylation mark gets lost. Changing the truth value from false to true is equivalent to applying a DNA methyltransferase that sets the methylation mark.

TABLE 3
Methylation logic table for the NOT operator.

| Boolean Logic | | MethyLogic | | |
|---|---|---|---|---|
| A | NOT A | A | NOT A | Realization |
| T | ⊥ | M | U | unmethylated ssDNA or unmethylated |
| | | | | at one site |
| ⊥ | T | U | M | Methylated ssDNA or methylated at |
| | | | | one site |

The following describes an exemplary use of methylation logic with Double Stranded DNA (dsDNA):

Logical Operator AND using dsDNA

The logical variables are encoded as double-stranded DNA. These strands can be ligated. The truth value of a ligated product is “True” if and only if the whole DNA sequence is methylated. Table 4 shows the Boolean logic and methylation logic equivalent for the logical operator AND.

TABLE 4
Methylation logic table for AND operator using dsDNA.

| Boolean Logic | | | MethyLogic | | | |
|---|---|---|---|---|---|---|
| A | B | A AND B | A | B | A AND B | Realization |
| T | T | T | M | M | M | fully methylated sequence |
| T | ⊥ | ⊥ | M | U | U | unmethylated at least on one |
| | | | | | | CpG |
| ⊥ | T | ⊥ | U | M | U | unmethylated at least on one |
| | | | | | | CpG |
| ⊥ | ⊥ | ⊥ | U | U | U | fully unmethylated sequence |

In certain embodiments of the present disclosure, implementation of an AND term may require an experimental procedure to verify for full methylation. The procedure should be capable of detecting unmethylation, even if a single C within the CpG dinucleotide is unmethylated. A bisulfite treatment may be used that will convert an unmethylated-C to a U, thus creating a mis-paired base with the G on the opposite strand. Those mis-paired bases can then be cut with the specific enzymes recognizing the mismatch. This protocol should yield only intact fully methylated DNA.

Logical Operator OR using dsDNA

The logical variables are encoded as double-stranded DNA then ligated. The truth value of the ligated product is equal to “True” if the double stranded DNA is methylated at least partially. Table 5 shows the Boolean logic and methylation logic equivalent for the logical operator OR using dsDNA. As in the case of AND, A and B are either ligated double-stranded DNA or two different subsequences on a longer double-stranded DNA sequence.

TABLE 5
Methylation logic table for OR operator using dsDNA.

| Boolean Logic | | | MethyLogic | | | |
|---|---|---|---|---|---|---|
| A | B | A OR B | A | B | A OR B | Realization |
| T | T | T | M | M | M | fully methylated |
| T | ⊥ | T | M | U | M | partially methylated |
| ⊥ | T | T | U | M | M | partially methylated |
| ⊥ | ⊥ | ⊥ | U | U | U | fully unmethylated |

In certain embodiments of the present disclosure, implementation of an OR term may require an experimental procedure to verify if a sequence is fully or partially methylated. Bisulfite sequencing is a method capable of checking for methylation of single sites. Alternatively, methyl binding proteins including methyl specific antibodies, or other separation technique can be used, as mentioned in implementation scenario 4, to fish out anything that has methylation. The unmethylated DNA would be in the unbound portion.

Logical Operator NOT using dsDNA

Table 6 shows the Boolean logic and methylation logic equivalent for the logical operator NOT. A logical variable is encoded as double stranded DNA. The truth value is reversed by using PCR if the sequence is methylated because during PCR the methylation mark gets lost. Changing the truth value from false to true is equivalent to applying a DNA methyltransferase that sets the methylation mark.

TABLE 6
Methylation logic table for the NOT operator using dsDNA.

| Boolean Logic | | MethyLogic | | |
|---|---|---|---|---|
| A | NOT A | A | NOT A | Realization |
| T | ⊥ | M | U | unmethylated dsDNA or unmethylated |
| | | | | at a particular position |
| ⊥ | T | U | M | Methylated dsDNA |

To further illustrate the uses and advantages associated with the disclosed systems and methods, reference is made to the following examples. However, it is to be understood that such examples are not limiting with respect to the scope of the present disclosure, but are merely illustrative of exemplary implementations and/or utilities thereof:

### EXAMPLE 1

Let p, q, r be Boolean variables and let p′, q′, r′, be their respective negations. Does there exist an assignment of truth values (T/F) to the variables p, q and r for which each of the four clauses p OR q, p′OR q OR r′, q′ OR r′, p′ OR r has the value true? In this example, the variables are encoded as three distinct double-stranded DNA and then ligated together. Here the methylated p (Mp) site is equated with p, the unmethylated p (Up) site with p′, Mq with q, Uq with q′ and Mr with r and Ur with r′. All possible combinations of these variables are created using ligation of the methylated and unmethylated double-stranded elements (MpMqMr, MpUqMr, MpMqUr, MpUqUr, ETC.). A vast amount of these molecules should be available. The variables will be defined so there exists a binding protein that can specifically bind to the methylated form of each variable. For these clauses, to save the p′ form, the DNA is applied to the methyl-binding protein and then the DNA that does not bind is saved (saves only the unmethylated or Up form). To save the p or methylated form, apply the DNA to the same protein, but save the bound DNA. Details for each clause are given below.

Step 1: Compute p OR q. (Illustrated in FIG. 1)

Separate the vast mixture of the starting DNAs into two pots.

In one, apply the mixture to the methyl-binding protein specific for the p site and in the other pot, apply the mixture to the methyl-binding protein specific for the q site.

In both cases, save the bound material, those that contain either Mp or Mq (p OR q). This sample would contain p OR q OR r and p OR q OR r′.

Recombine these two bound samples. This mixture now contains 6 different double-stranded DNAs: MpUqMr, MpUqUr, UpMqMr, UpMqUr, MpMqMr, and MpMqUr.

Step 2: Compute p′ OR q OR r′. (Illustrated in FIG. 12)

Separate the mixture of the DNA from the last step into three pots.

In one, apply the mixture to the methyl-binding protein specific for the p site and save the unbound material. In another pot, apply the mixture to the methyl-binding protein specific for the q site and save the bound material. In the third pot, apply the mixture to the methyl-binding protein specific for the r site and save the unbound material.

Recombine the three saved samples. This sample now contains MpUqUr, UpMqMr, UpMqUr, MpMqMr and MpMqUr.

Step 3: Compute q′ OR r′.

Separate the mixture of DNA from the last step into two pots.

In one, apply the mixture to the methyl-binding protein specific for the q site and save the unbound material. In the other pot, apply the mixture to the methyl-binding protein specific for the r site and save the unbound material.

Recombine the two saved samples. This sample now contains MpUqUr, UpMqUr and MpMqUr.

Step 4: Compute p′ OR r

Separate the mixture of DNA from the last step into two pots.

In one, apply the mixture to the methyl-binding protein specific for the p site and save the unbound material. In the other pot, apply the mixture to the methyl-binding protein specific for the r site and save the bound material.

Recombine the two saved samples. This sample should only contain UpMqUr from the bound material from the methyl-p site binding protein. The unbound material from the methyl-r binding protein will yield no DNA as all the molecules from the previous step contain Mr.

Step 5: Read the answer

Apply bisulfite treatment to the material and sequence the resulting DNA fragments. Bisulfite treatment converts unmethylated Cs to Us while has no effect on methylated Cs. Where the sequence is the same as the starting material, that site was methylated in the final product. Where the sequence is different and a U is substituted for a C, that site was unmethylated in the final answer.

### EXAMPLE 2

Represent a logical formula: (a OR b) AND (c′) AND d using MethyLogic. It can be thought of also as a representation of a logic circuit. The goal is to know for which inputs (values of a, b, c and d) the logic circuit produces a “true” value. Here a representation of logical variables with single-stranded DNA is used.

Step 1: Compute a OR b

A is encoded with a sequence and then b with another sequence in such a way that they would hybridize. For example, a would be encoded with 5′-ACGCGA-3′ then b encoded with 5′-AAATCG-3′. The hybridized form of this DNA would be represented as below: (a more than a 3 base overlap for better hybridization is preferred). It should also be noted that all sequences need to contain at least one C so it can be methylated. One can also work with methylated As if necessary.

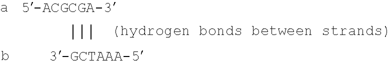

Combine four pots containing unmethylated a (Ua), methylated a (Ma), Ub, and Mb and create four different kinds of double stranded DNA (Ua/Ub, Ua/Mb, Ma/Ub, and Ma/Mb).

Use methyl-binding proteins (e.g. MeCP or MBD1 or antibodies to methyl-C) to fish out the sequences that have hybridized and at the same time contain methylated Cs. This corresponds to performing a OR b.

Step 2: Compute c′ AND d

2.1 Encode c and d with different sequences in such a way that they would hybridize together, and as a hybrid ligate with the overhang of the a OR b hybrid (see below). For example, c could be encoded with 5′-TTTGCG-3′ then d would be encoded with 5′-ATACGC-3′ such that when hybridized they form a structure as below: (more than a 3-base overlap for better hybridization is preferred).

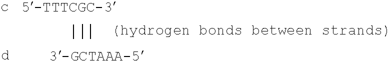

As above, create 4 types of single-stranded DNAs, Uc, Ud, Mc and Md. 2.2 Apply NOT to c by applying PCR to the methylated c pot and apply methyltransferase to the unmethylated c pot. For simple variables pots can just be exchange.

2.3 Hybridize the two results with unmethylated d and methylated d.

2.4 Apply AND operator to the four pots: bisulfite treatment followed by enzyme that destroys mismatched DNA. The bisulfite treatment converts unmethylated Cs to U and does not affect the methylated Cs. Thus in a hybrid where an unmethylated C is hybridized with a G, following bisulfite treatment, it will yield a U-G mismatch instead of the normal C:G basepair. The mismatched DNA can be destroyed using specific enzymes that recognize the mismatched double stranded DNA.

Step 3: Compute the AND of the product from the previous two computations by combining the pots resulting from step 1 and 2 and ligate. The product of this reaction would have the DNA sequence structure as below:

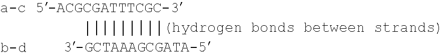

Apply bisulfite treatment followed by the enzyme that destroys mismatched DNA. As mentioned above, the bisulfite treatment should convert unmethylated Cs to Us and therefore generate a mismatched DNA sequence where U is opposite a G. Methylated Cs will not be modified by this treatment and therefore should remain correctly basepaired with Gs on the other strand. Mismatched DNA can then be destroyed using specific enzymes. The resulting DNA should satisfy the complex clause: a OR b AND c′ AND d.

Step 4: Read the answer. For this, divide the mixture into two pots and treat one of them with bisulfite. As mentioned above, this treatment converts unmethylated C's to U's. Then sequence the DNA strands in each pot. Sequence both strands in order to find the truth values of the logical variables in the circuit. Any difference will be because of unmethylated C at that position. In present case, the state of site c should be negated when reading the answer.

In a preferred application of the present disclosure, the MethyLogic method is used to first define the clauses in silico (this is equivalent to hypothesis generation in computer simulation) and then tested in vitro. A set of genes can be represented using logical variables, each logical variable representing a single gene or a specific site or sites on a gene or set of genes. The state of methylation of a gene's promoter, first exon, or any regulatory region, represents the truth value for that sequence. The samples come from control (i.e., healthy) and diseased (e.g., cancer) individuals. The problem is the same as in SAT problems: for which values of the logical variables (genes) do the clauses evaluate to true (distinguishing control from disease samples)? The truth value of the variables will indicate the biomarkers responsible for the healthy versus diseased samples. The disclosed systems and methods introduce novel and powerful ways to search for a set of clauses that can validate existing methylation biomarkers, as well as for finding new biomarkers. Overall, the present disclosure describes systems and methods to be used in combination with in-vitro and in-silico methods to assist in clinical environments.

The present disclosure describes and illustrates methods and systems of implementing Boolean logic with DNA methylation using both single and double stranded DNA. The examples described herein offer exemplary techniques/applications that can be used in a wide ranging implementations of a universal DNA computer based on “methylation logic”. This approach is more viable, dynamic and versatile than past approaches. In previous approaches, the presence of appropriate restriction enzymes and, recently methylases were always required. The prior approach inevitably limits the scope of DNA computing and types and numbers of sequences capable of being analyzed. The present paradigm should allow the potential analysis of almost any sequence, even one within a mixture as complex as the human genome. This method can be a great tool in decision making for molecular diagnostics.

In sum, the systems and methods of the present disclosure offer significantly enhanced techniques for DNA computation, particularly for biomarking and theoretical computation. Although the present disclosure has been described with reference to exemplary embodiments and implementations thereof, the disclosed systems and methods are not limited to such exemplary embodiments/implementations. Rather, as will be readily apparent to persons skilled in the art from the description provided herein, the disclosed systems and methods are susceptible to modifications, alterations and enhancements without departing from the spirit or scope of the present disclosure. Accordingly, the present disclosure expressly encompasses such modification, alterations and enhancements within the scope hereof.

1. A method using methylation logic comprising:
generating a logic statement having a plurality of assigned variables, wherein said plurality of variables includes a methylated variable and a negation of said methylated variable corresponding to an unmethylated variable,
methylating a mixture or sample;
reversing methylation status of a sample;
separating said mixture or sample through one or more separation techniques to isolate at least one separated mixture;
decoding the at least one separated mixture; and reading the at least one separated mixture to correlate said logic statement to said decoding.

2. A method according to claim 1, wherein said method is effective to verify biomarker(s) and/or create or identify a new biomarker.

3. A method according to claim 1, wherein the one or more separation techniques includes a series of assays.

4. A method according to claim 1, wherein the reading is effective to verify an existing biomarker or create/identify a new biomarker.

5. A method according to claim 1, wherein said plurality of variables are specified genes.

6. A method according to claim 1, wherein said plurality of variables are a set of genes.

7. A method according to claim 1, wherein said plurality of variables are a set of sites on a gene.

8. A method according to claim 1, wherein said plurality of variables has at least one cytosine for a C-implementation methylation encoding.

9. A method according to claim 1, wherein said plurality of variables has at least one adenine for an A-implementation methylation encoding.

10. A method according to claim 1, wherein said plurality of variables are encoded using single-stranded DNA.

11. A method according to claim 1, wherein said plurality of variables are encoded using double-stranded DNA.

12. A method according to claim 1, wherein said plurality of variables are encoded using methyl binding proteins.

13. A method according to claim 1 further comprises generating logic circuits.

14. A system for solving a set of genetic clauses comprising
assigning variables, wherein each variable corresponds to a methylated variable and negation of said variable corresponding to an unmethylated variable,
solving for AND, OR, and NOT Boolean logic terms within a given logic clause utilizing said methylated and unmethylated variables by:
methylating a given mixture of said variables, reversing methylation status of a sample;
separating said mixture to yield a desired mixture satisfying said given logical clause.

15. A method according to claim 12, wherein said variables are specified genes.

16. A method according to claim 12, wherein said variables are a set of genes.

17. A method according to claim 12, wherein said variables are a set of sites on a gene.

18. A method according to claim 12, wherein at least one of said variables has at least one cytosine for a C-implementation methylation encoding.

19. A method according to claim 12, wherein at least one of said variables has at least one adenine for an A-implementation methylation encoding.

20. A method according to claim 12, wherein said variables are encoded using single-strand DNA.

21. A method according to claim 12, wherein said variables are encoded using double-strand DNA.
